(19) United States
(12) Patent Application Publication
(10) Pub. No.: US 2023/0257692 A1
(43) Pub. Date: Aug. 17, 2023

(54) FILTRATION ASSEMBLIES, CASSETTES, SYSTEMS, AND METHODS FOR FILTRATION AND CELL GROWTH

(71) Applicant: RAPID MICRO BIOSYSTEMS, INC., Lowell, MA (US)

(72) Inventors: John RIPPETH, Melbourn, Herts (GB); Max WEHEBRINK, Melbourn, Herts (GB); Frank TYLDESLEY, Melbourn, Herts (GB); Richard SMITH, Melbourn, Herts (GB)

(21) Appl. No.: 18/004,589

(22) PCT Filed: Jul. 8, 2021
(86) PCT No.: PCT/US2021/040935

§ 371 Date: Jan. 6, 2023

Related U.S. Application Data
(60) Provisional application No. 63/049,513, filed on Jul. 8, 2020.

(51) Int. Cl. C12M 1/26; C12Q 1/24; C12M 1/00; C12M 3/00

(52) U.S. Cl. CPC C12M 33/14; C12Q 1/24; C12M 23/02; C12M 23/42; C12M 23/48

(57) ABSTRACT

The present invention features filtration assemblies, cassettes, systems, and methods for filtering cells from a sample solution and then incubating the captured cells.

### BACKGROUND OF THE INVENTION

Many industries require the determination of the number of microbes in a sample. One method of determining the number of microbes in a sample involves capturing microbes on a membrane, culturing the microbes on the membrane, and counting the number of colonies formed. Manipulation of the membrane can introduce non-sample microorganism contaminants or debris, damage the membrane, or create defects, all of which can frustrate enumeration.

Thus, there is a need for new filtration and culturing devices for microbial enumeration.

### SUMMARY OF THE INVENTION

The invention provides devices, systems, and kits for filtering cells from a sample solution and then culturing the captured cells for, e.g., imaging and enumeration of colony forming units (CFUs). Filtration assemblies, cassettes, systems, and methods of the invention are particularly amenable to incorporation into automated enumeration systems and processes.

In a first aspect, the invention provides a filtration assembly, including a funnel, a membrane frame including a porous membrane, and a base with a membrane support and an outlet. The membrane frame is releasably attached to the funnel, and the base is releasably attached to the funnel. During filtration, liquid flows from the funnel through the membrane and membrane support to the outlet. In some embodiments, the membrane support keeps the membrane flat during filtration.

In some embodiments, the funnel is attached to the base by a locking mechanism that releases when a portion of an exterior wall of the base is pressed. In certain embodiments, the porous membrane is ultrasonically bonded or heat staked to the membrane frame. In other embodiments, the membrane frame is releasably attached to the funnel by an interlocking arrangement of a raised lip on the funnel and a recess in the membrane frame. In certain embodiments, the membrane frame includes a protruding ring that interlocks with tabs on a cassette base. In some embodiments, the membrane is black and/or non-fluorescent. In particular embodiments, the membrane is a black, mixed cellulose ester membrane.

In some embodiments, the funnel is transparent and/or includes volumetric markings. In certain embodiments, the filtration assembly also includes a funnel lid that covers an opening to the funnel. In some embodiments, the funnel lid is hinged on the funnel. In some embodiments, the membrane support is attached to the filter base. In certain embodiments, the filter base includes a plurality of supports configured to promote fluid flow to the outlet and/or to support the membrane support. In some embodiments, the membrane frame may also include fiducial markings.

In some embodiments, the filtration assembly further comprises a washer. In certain embodiments, the washer is attached to the membrane support and/or is a thin film. In particular embodiments, the washer and membrane support are a single molded part. In some embodiments, the washer comprises a spring. In some embodiments, the washer and membrane support are a single molded part.

In certain embodiments, the filtration assembly includes a cavity and/or a shim between the membrane and membrane support. In certain embodiments, the membrane support and/or the base are shaped to create the cavity.

A second aspect of the invention provides a cassette base, with a base layer including an outer wall, a first inner wall including a plurality of radially disposed gaps, and a second inner wall. The second inner wall has an outside ledge and defines a well, and the first inner wall is disposed between the outer wall and the second inner wall. The cassette also includes a solid or semi-solid nutrient media in the well. The media has a flat growth area that is higher than the second inner wall. The cassette also includes a cassette lid releasably sealable to the base.

In some embodiments, the cassette lid is optically transparent and non-fluorescent. In some embodiments, the cassette base is non-fluorescent. In certain embodiments, the cassette further includes a third inner wall disposed between the first inner wall and the outer wall. In particular embodiments, the cassette also includes a fourth inner wall between the third inner wall and the outer wall.

In some embodiments, the second inner wall includes a plurality of supports protruding radially towards the first inner wall.

In some embodiments, the cassette includes a washer. In certain embodiments, the washer is attached to the cassette base and/or is a thin film. In particular embodiments, the washer and cassette base are a single molded part. In some embodiments, the washer includes a spring.

In some embodiments, the cassette base is disposed to receive a membrane lowered onto the base at an angle between 1° and 75° relative to the flat growth area. In certain embodiments, the cassette base includes a ring supported by an annularly arranged plurality of springs, where a highest spring of the plurality of springs is antipodal to a lowest spring of the plurality and springs of the plurality disposed therebetween are tapered in height. In some embodiments, the cassette base includes a feature that engages the membrane frame to result in an initial angle between the membrane and the flat growth area of between 1° and 75°.

Another aspect of the invention provides a system for filtering and culturing cells. The system can include an embodiment of the filtration assembly the first aspect, and an embodiment of the cassette of the second aspect. The membrane frame is configured to detach from the filter base with the funnel, to attach to the cassette base, and to detach from the funnel once attached to the cassette base.

In some embodiments of the system, the membrane frame includes a protruding ring, and the cassette base includes tabs that interlock with the protruding ring. In some embodiments, attaching the membrane frame to the cassette base places the membrane in conformal contact with the flat growth area.

A further aspect of the invention provides a method for determining the presence of microorganisms. The method includes attaching the filtration assembly of any embodiment of the first aspect to a source of vacuum. The method further includes flowing a sample fluid through the filtration assembly, such that any cells in the fluid are retained on the membrane, detaching the funnel attached to the membrane frame from the base, attaching the membrane frame to a cassette base of the second aspect, and detaching the funnel from the membrane frame. The membrane may then be incubated.

In some embodiments, the method further includes imaging the membrane to detect any colonies formed from the retained cells.

In some embodiments, the filtration assembly includes a cavity, and the method includes applying pressure, e.g., from vacuum, to cause the membrane to conform to a shape of the membrane support and/or cavity. In certain embodiments, the membrane maintains the shape of the membrane support and/or cavity after detaching the funnel from the base. In particular embodiments, the shaped membrane interacts with the cassette to prevent bubble trapping during attachment of the membrane from to the cassette base.

In some embodiments, the membrane makes first contact with the flat growth area at a first point on its circumference and a final contact at a second point on the circumference that is antipodal to the first point. In some embodiments, the membrane frame is initially lowered onto the base at an angle between 1° and 75° relative to the flat growth area. In certain embodiments, the cassette includes a feature that engages the membrane frame to position the membrane to contact the flat growth area at an angle between 1° and 75° relative to the flat growth area as the membrane frame is lowered onto the cassette base. In certain embodiments, the features include a tapered a ring supported by an annularly arranged plurality of springs, where a highest spring of the plurality of springs is antipodal to a lowest spring of the plurality and springs of the plurality disposed therebetween are tapered in height.

It will be understood that the filtration assemblies, cassettes, systems, and methods described herein may include additional features beyond those here specified, including any that are not inconsistent with the structure of the underlying filtration assembly, cassette, system, or method.

The term “about,” as used herein, refers to ±10% of a recited value.

### BRIEF DESCRIPTION OF THE DRAWINGS

FIG. 1 is an illustration showing filtration of a sample then transfer of the membrane to a cassette base for cell growth.

FIG. 2A is a schematic drawing showing a funnel of the invention with a lid. FIG. 2B is a schematic showing an exploded view of a filtration assembly of the invention, featuring: a funnel with a funnel lid; a membrane frame with a membrane, and a base.

FIG. 3 is a schematic drawing showing examples of (left to right): a membrane frame without the membrane; a filtration assembly base and membrane support (e.g., a flat sintered thermoplastic polymer disc, such as Porex®); and a cassette base.

FIG. 4 is a schematic drawing showing a cross-section of a funnel, membrane frame, and base of the invention when attached.

FIG. 5 is a schematic drawing showing a close-up of a funnel, membrane frame, and base.

FIG. 6 is a schematic drawing of an embodiment of the invention featuring a transparent funnel with volumetric markings and a base featuring a push button in the exterior wall of the base for releasing the funnel from the base.

FIG. 7A is a schematic drawing of filtration assemblies of the invention stacked without a lid. FIG. 7B is a schematic of filtration assemblies of the invention stacked with lids.

FIG. 8A is a schematic drawing of a filtration assembly of the invention featuring an injection molded lid.

FIG. 8B is a schematic of a filter of the invention featuring a thermoformed lid.

FIG. 9 is an illustration showing a filtration assembly of the invention with three different lids.

FIG. 10 is a schematic drawing showing a filtration assembly of the invention having hinged lid.

FIGS. 11A-11B are schematic drawings showing staggered arrangement of filtration assemblies of the invention.

FIGS. 12A-12B are schematic drawings showing staggered arrangement of filtration assemblies of the invention compared to a rectangular arrangement.

FIGS. 13A-13B are schematic drawings showing staggered arrangement of filtration assemblies of the invention on a tray of the invention.

FIG. 14 is a schematic drawing showing a cross-section of a filtration assembly with funnel, membrane frame, and base when attached.

FIG. 15A is a schematic drawing showing top and bottom views of a funnel of the invention. FIG. 15B is schematic drawing showing top and bottom views of a base of the invention. FIG. 15C is schematic drawing showing top and bottom views of a membrane frame (without membrane) of the invention.

FIG. 16A is a schematic drawing showing top and bottom views of a cassette base of the invention. FIG. 16B is a schematic drawing showing a cross section of a funnel, membrane frame, and cassette base of the invention when attached.

FIG. 17 is a schematic drawing showing a cross-section of a filtration assembly with funnel, membrane frame, and base when attached.

FIG. 18A is a schematic drawing showing top and bottom views of a funnel of the invention. FIG. 18B is schematic drawing showing top and bottom views of a base of the invention. FIG. 18C is schematic drawing showing top and bottom views of a membrane frame (without membrane) of the invention.

FIG. 19A is a schematic drawing showing top and bottom views of a cassette base of the invention. FIG. 19B is a schematic drawing showing a cross section of a funnel, membrane frame, and cassette base when attached.

FIG. 20 is a schematic drawing showing a close-up cross-section of a funnel, membrane frame, and cassette base when attached.

FIG. 21 is a schematic drawing showing a close-up cross-section of a portion of a filtration assembly, including funnel, membrane frame, and base drawing when attached, and showing a seal and various tabs.

FIG. 22 is a schematic drawing showing a close-up cross-section of a funnel, membrane frame, and cassette base of the invention, including a feature on the cassette base for disengaging a tab on the membrane frame.

FIG. 23 is an illustration of a cassette base of the invention with solid or semi-solid nutrient media and a membrane in a membrane frame, showing the membrane in the membrane frame being placed on the solid or semi-solid nutrient media at an angle (top to bottom).

FIG. 24 is a schematic drawing showing a cross-section of a filtration assembly of the invention.

FIG. 25 is an illustration showing a cassette base of the invention with solid or semi-solid nutrient media and a washer.

FIG. 26A is an illustration showing a partial cross section of the invention during the filtration steps of a method of the invention. FIG. 26B is an illustration showing attachment of a membrane in a membrane frame to a cassette base.

FIG. 27 is an illustration showing various beneficial features of the invention.

FIGS. 28A-28D are illustrations showing different washers of the invention. FIG. 28A shows a washer as a part attached (e.g., by mechanical interengagement) to the membrane ring. FIG. 28B shows the washer as a thin film attached (e.g., by mechanical interengagement) to the membrane frame. FIG. 28C shows the washer and membrane frame as a single molded part. FIG. 28D shows the washer as a thin film joined (e.g., thermally) to the membrane frame.

FIGS. 29A-29C are illustrations of washers of the invention that are attached to the cassette base. FIG. 29A shows the washer as an attached part in contact with the solid or semi-solid nutrient media. FIG. 29B shows the washer as a thin film attached to the cassette and in contact with the solid or semi-solid nutrient media. FIG. 29C shows the washer as a spring attached to the cassette.

FIGS. 30A and 30B are photographs of a membrane of the invention on a filtration assembly base of the invention having an angled shim.

FIG. 31 is a photograph of a cassette base of the invention including an angled sprung element to control membrane laydown during transfer of the membrane to the cassette base.

FIG. 32 is a schematic drawing showing a partial cut-out view of a filtration assembly of the invention including a washer of the invention.

FIG. 33 is a schematic drawing showing a cross-section of a cassette base of the invention with a membrane in a membrane frame of the invention with thin film (e.g., 0.1 mm) and thicker (e.g., 0.45 mm) washers of the invention before and after laydown of the membrane on the solid or semi-solid nutrient media in the cassette base. The thin film (e.g., 0.1 mm thickness) conforms to the membrane pressure. The thicker (e.g., 0.45 mm) washer conforms to the membrane pressure and takes on a Belleville washer shape and digs into solid or semi-solid nutrient media. An even thicker (e.g., 0.80 washer) is rigid and does less conforming to the membrane under pressure leading to extra stretch and digs in to solid or semi-solid nutrient media.

### DETAILED DESCRIPTION OF THE INVENTION

The invention provides filtration assemblies, cassettes, systems, and methods for microbiological growth enumeration, e.g., bacteria, fungi, and archaea. The devices, systems, and methods of the invention are particularly amenable to automated enumeration and allow transfer of membranes retaining cells between components in a manner that reduces the possibility of damage to, or contamination of, the membrane.

### Filtration Assembly

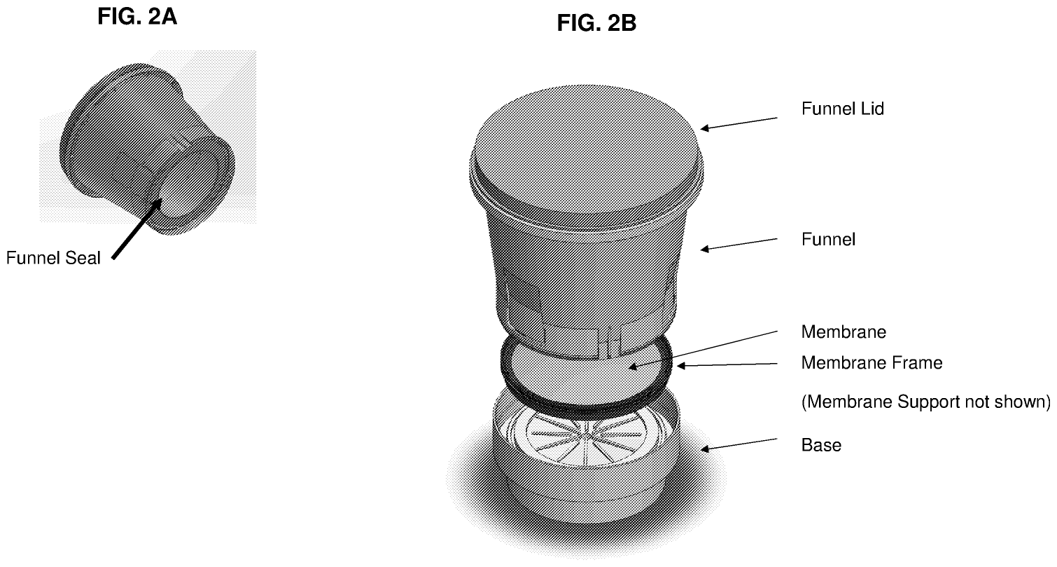

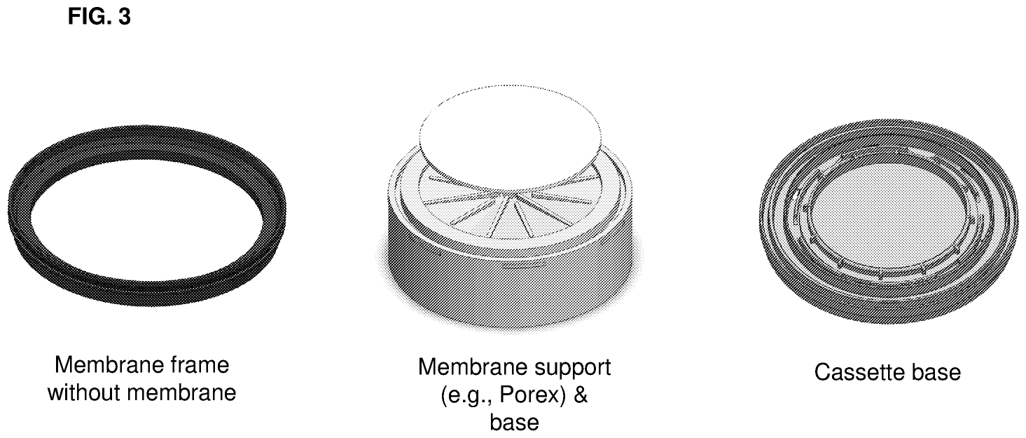

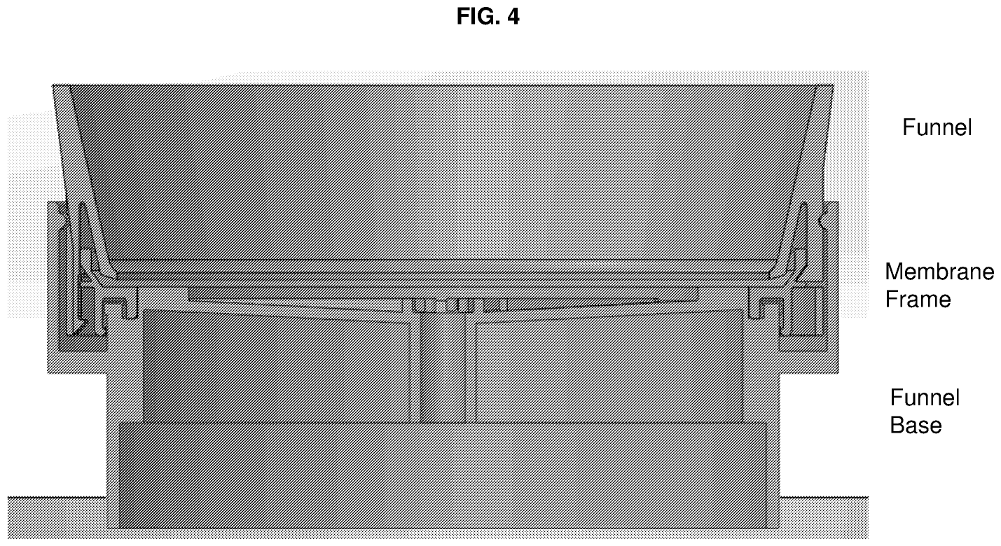

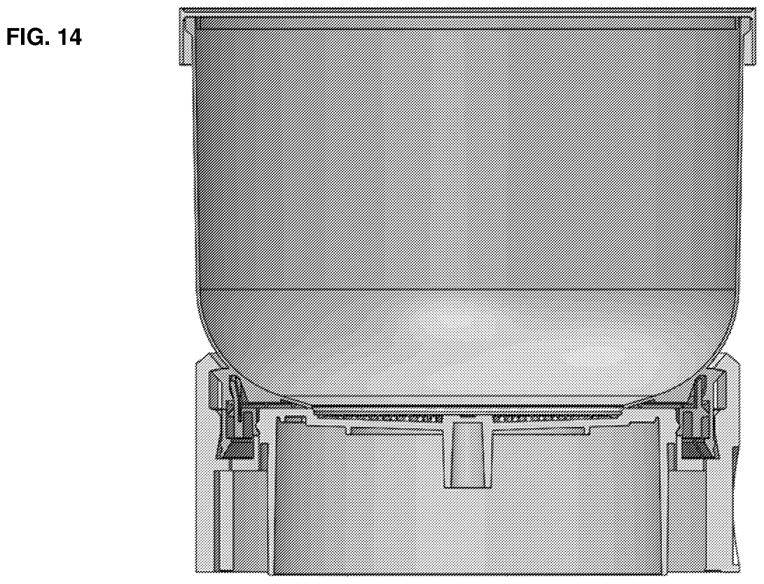

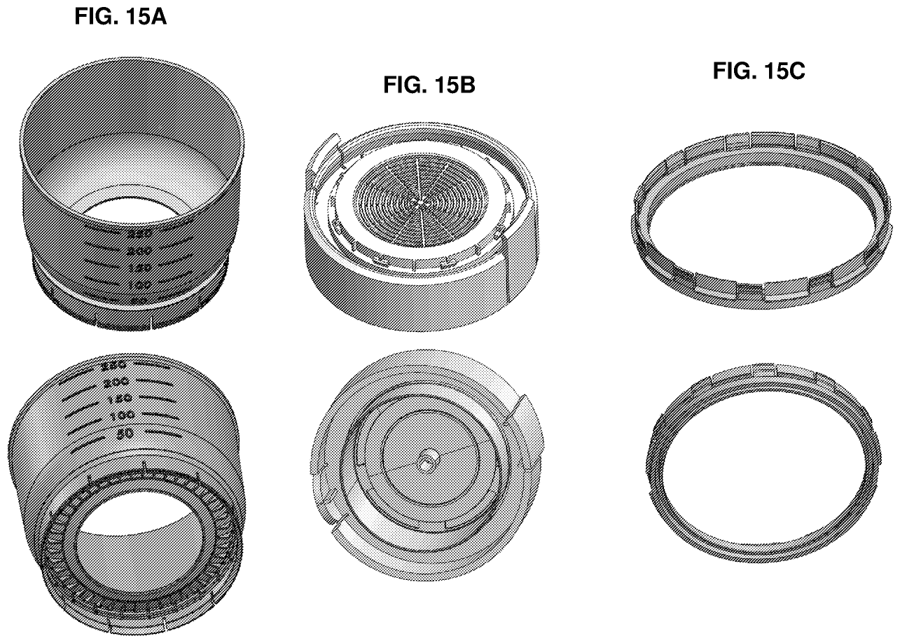

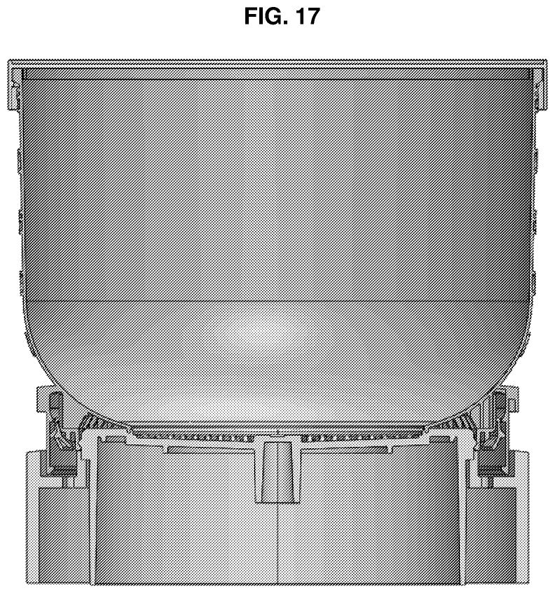

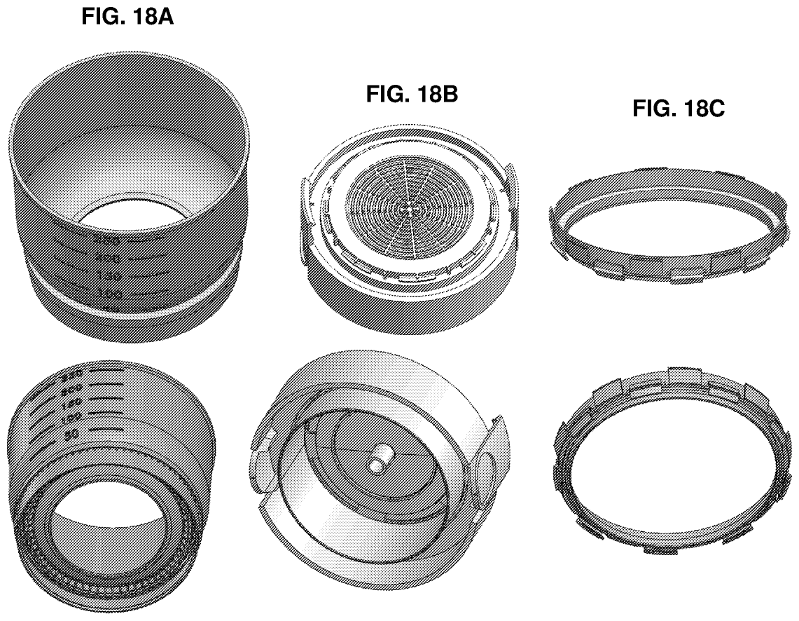

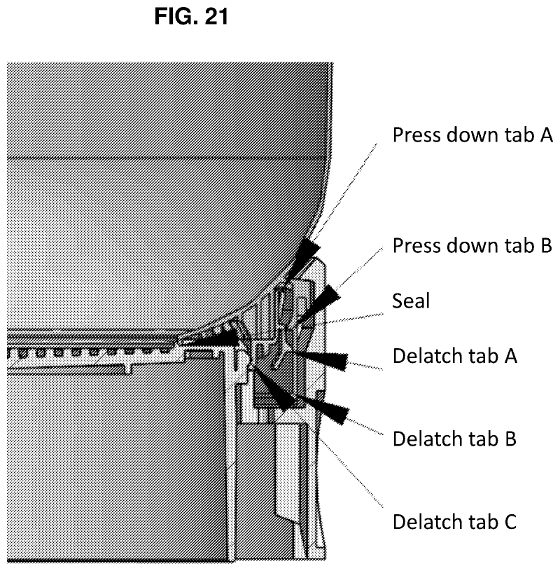

One device of the invention is a filtration assembly for filtering a sample and retaining any microbes that may be found therein (see, e.g., FIG. 2B, FIG. 14, or FIG. 17). A filtration assembly of the invention features a funnel (e.g., FIG. 2A, FIG. 15A, or FIG. 18A), a membrane frame (e.g., a ring, as shown in FIG. 3, FIG. 15C, or FIG. 18C) that includes a porous membrane (e.g., a mixed cellulose ester membrane), and base (e.g., FIG. 3, FIG. 15B, or FIG. 18B) with a membrane support (e.g., a flat disc, or shaped disc with a flat portion, of sintered thermoplastic polymer) and an outlet (e.g., for connection to a source of vacuum). The membrane frame is releasably attached to the funnel (e.g., by interlocking or overlapping features or friction or jam fit, see, for example, FIG. 4 or FIG. 21), and the base is releasably attached to the funnel (e.g., with a locking mechanism). During filtration, liquid flows from the funnel through the membrane and membrane support to the outlet. The structure of the assembly allows the membrane frame to be detached from the base while attached to the funnel for transfer to a cassette base in a manner that avoids directly handling the membrane or membrane frame.

In some embodiments, the membrane support keeps the membrane flat during filtration, for example, by having a height that keeps the membrane support and the membrane in conformal contact. Suitable materials that can be sintered to produce the membrane support include, e.g., high density or ultra-high molecular weight polyethylene, polyether sulfone, polypropylene, polytetrafluoroethylene, polyvinylidene fluoride, etc. Other thermoplastic polymers known in the art may also be used. In some embodiments, the membrane support is attached to the filter base (e.g., by gluing or interlocking features), which prevents the membrane support from being removed with the funnel and membrane frame. The membrane support may also be incorporated directly into the base, e.g., during manufacturing of the base. Alternatively, the membrane support allows the membrane to deform during filtration, e.g., to conform to a cavity in the membrane support.

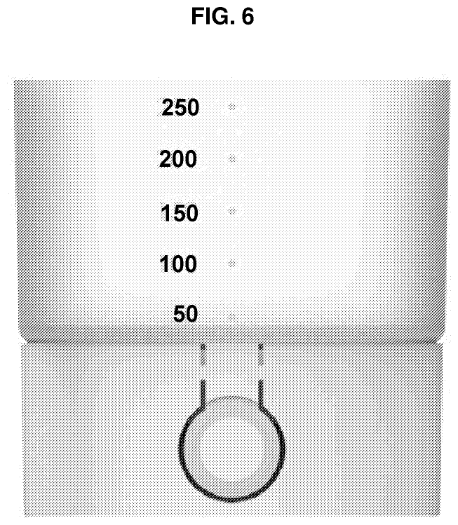

In some embodiments, the funnel is attached to the base by a locking mechanism that releases when a portion of an exterior wall of the base is pressed (e.g., a button that disengages a latch, e.g., FIG. 6). In one example, the portion of the exterior of the wall of the base may include a flexible hinge with an inward protrusion (e.g., a tab), which latches an outward protruding feature on a portion of an exterior wall of the funnel (e.g., a ring or ledge), where pressing the hinge retracts the latch, releasing the funnel from the base. The hinge may also feature a fulcrum. Other suitable locking mechanisms include deformable tabs or catches which can be separated by pulling the funnel and base apart.

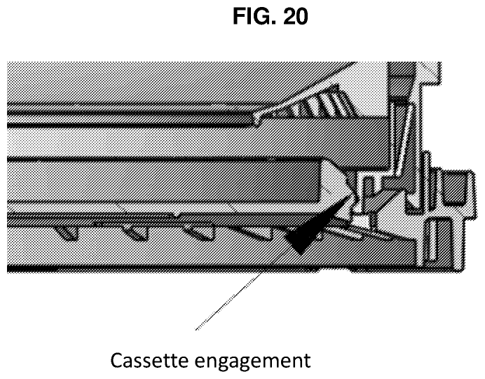

In certain embodiments, the porous membrane is ultrasonically bonded or heat staked to the membrane frame. Other suitable bonding methods may include adhesive bonding, mechanical retention, etc. The membrane frame may be releasably attached to the funnel by, for example, an interlocking arrangement of a raised lip on the funnel and a recess in the outer wall of the membrane frame (e.g., FIG. 4). In certain embodiments, the membrane frame can include a protruding ring that interlocks with tabs on a cassette base. Alternatively, the tabs may be on the membrane frame (e.g., FIG. 15C, or FIG. 18C) and the ring on the cassette base (e.g., FIG. 20). Locking features (e.g., tabs or rings) on the membrane frame can be configured to more strongly hold the membrane frame to the funnel than to the base of the filtration assembly. Locking features on the membrane frame may be the same features for attachment to the base of the filtration assembly as the cassette base, but with the complementary feature, or features, of the filtration assembly base providing a weaker interaction than the corresponding feature, or features, on the cassette base. Locking features (e.g., tabs) on the membrane frame may also be configured to interact with features on the cassette base which act to disengage the locking feature from the funnel. Locking features on the membrane frame may be antipodally non-symmetric, e.g., configured to engage with the cassette base first with one locking feature and last with a locking feature that is antipodal to the first locking feature.

The membrane frame includes a porous membrane on which cells are retained during filtration (see, e.g., FIG. 2B). The membrane may be black and/or non-fluorescent, so as to not interfere with imaging and enumeration. An exemplary membrane is a black mixed cellulose ester membrane. Other suitable membrane materials may include, e.g., cellulose, cellulose acetate, ethylene vinyl acetate, polystyrene, nitrocellulose, polyether ether ketone, Nylon, polyolefin (e.g., polyethylene or polypropylene), polyacrylonitrile, polyethylene terephthalate (PET), polyethersulfone, track-etched polyester or polycarbonate, polyvinylidene fluoride, polytetrafluoroethylene, cellulose acetate, and silicone copolymer, etc. Membranes may also feature surface coatings, e.g., to allow or promote cell attachment or colony growth. The choice of material and/or coating may depend on the type of cells that are expected to be retained. Membranes of the invention have pore diameters sized to prevent the passage of microorganisms such as bacteria or yeasts, e.g., about 0.45 μm, e.g., about 0.1-1 μm (e.g., 0.1 μm, 0.2 μm, 0.3 μm, 0.4 μm, 0.5 μm, 0.6 μm, 0.7 μm, 0.8 μm, 0.9 μm, or 1 μm), depending on the application. The membrane frame outside of the membrane may be made of any suitable material that allows for attachment to the filter and the base, e.g., plastic or metal.

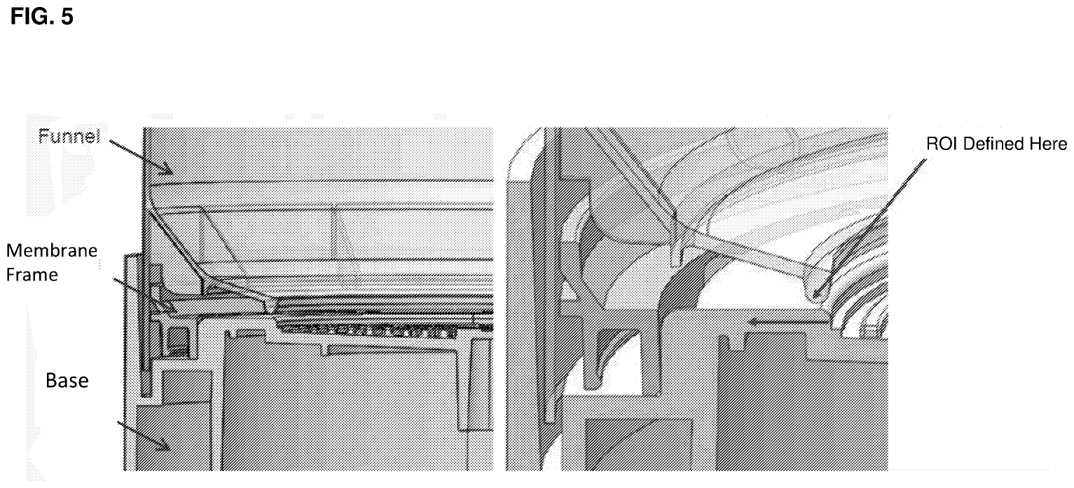

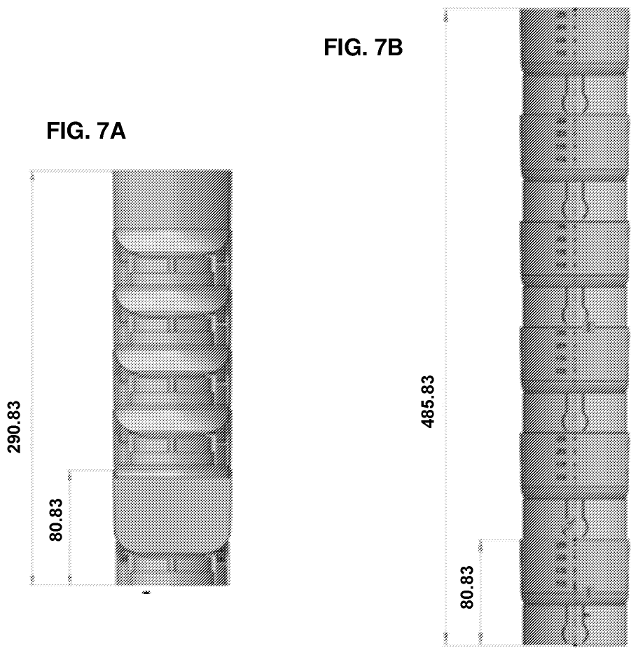

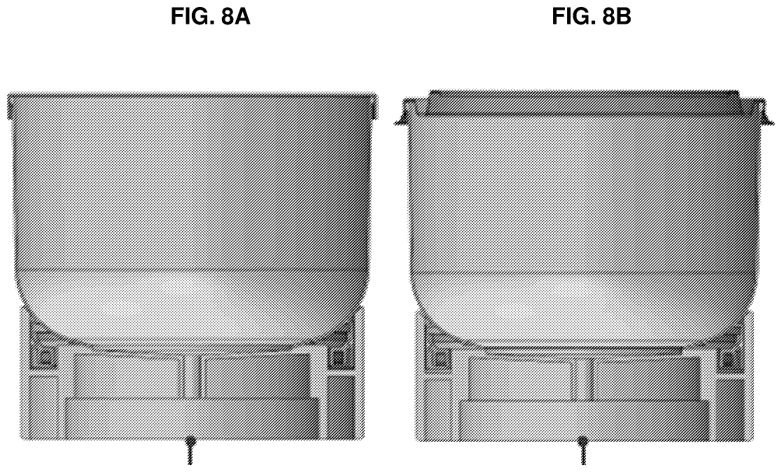

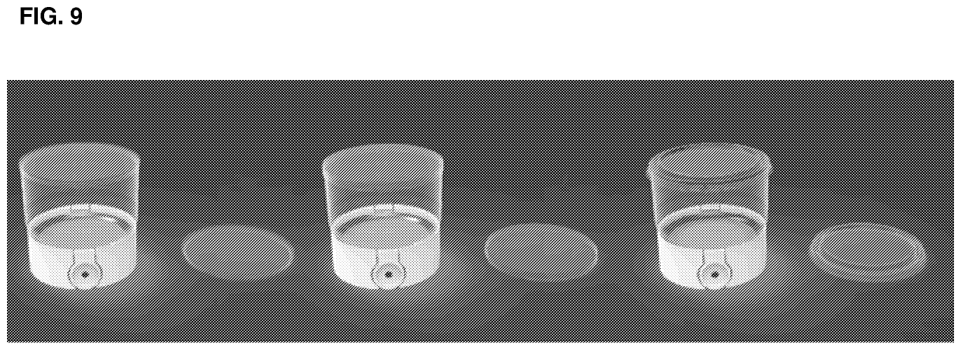

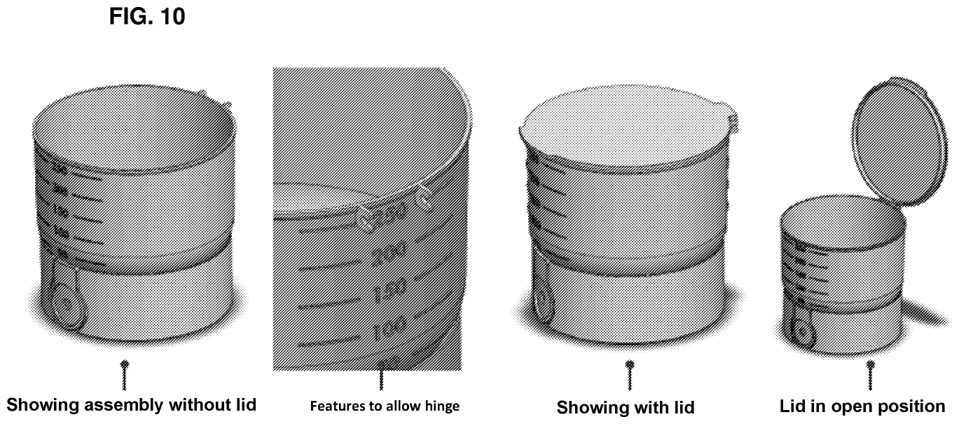

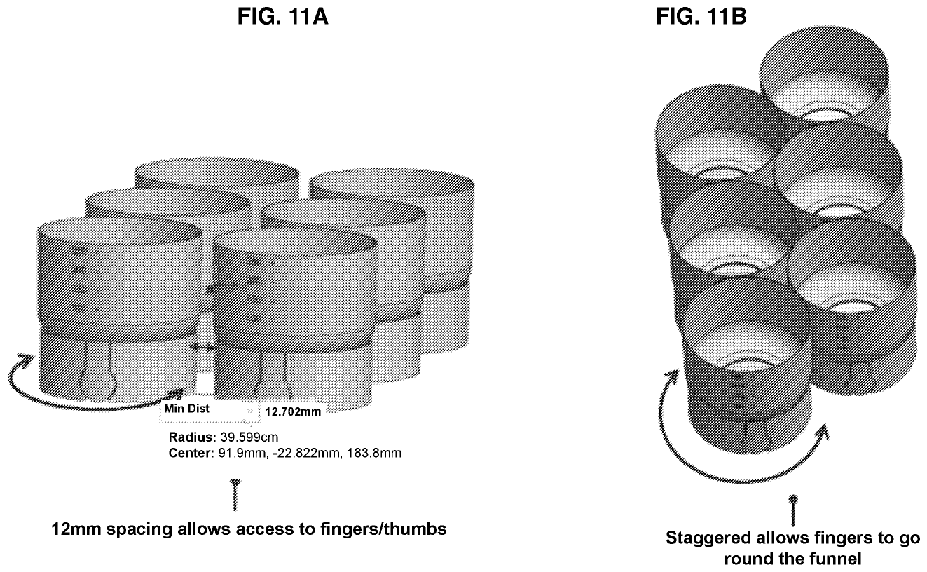

The funnel may be transparent and/or include volumetric markings to assist the user in adding the correct quantity of sample fluid, and to allow for volumetric data generation, i.e., number of CFUs per unit volume of sample fluid. The funnel may also include a seal (e.g., FIG. 2A) in the interior that presses against the membrane and defines a region of interest (e.g., FIG. 5), which defines the area where cells may be found. When the funnel and membrane frame are removed from the base, the membrane may separate from the seal, e.g., to prevent the seal from interacting with the nutrient media when the membrane frame is attached to the cassette base. The filtration assembly may also include a funnel lid that covers an opening to the funnel, which may be hinged on the funnel itself. The funnel lid can act to protect the membrane surface from contamination during storage or use. A hinged lid may be formed from a separate lid and funnel, for example, by each of the lid and funnel having features that clip together to form the hinge (see, e.g., FIG. 10). Such a set-up has advantages for storage and transport of multiple filtration assemblies, as they can be more efficiently stacked without a lid in the cover position (see, e.g., FIG. 7A to FIG. 7C). Lids of the invention may be (e.g., FIG. 8B or FIG. 9) thermoformed or injection-molded (e.g., FIG. 8A or FIG. 9) and may be of the same material of the funnel or another suitable material. The funnel and lid may be formed of any suitable material such as plastic or metal.

The base connects the filtration assembly to a source of vacuum, e.g., via a tulip. A base of the invention may be shaped to couple to, e.g., a tulip valve, allowing fluid communication of the outlet to the source of vacuum. In certain embodiments, the filter base includes a plurality of supports configured to promote fluid flow to the outlet and/or to support the membrane support. The filtration base may include features for mating with the source of vacuum, for example, grooves to accept the lip of a tulip valve, or another sealing member.

Since the end use of the membrane frame may be for imaging of colony growth, the membrane frame or membrane may also include fiducial markings to help maintain alignment of multiple images over, e.g., an incubation time series. Fiducial markings may also assist in alignment with robotic handling systems.

The membrane frame may be configured (e.g., by having positioning features) to be initially lowered onto the base at an angle between 1° and 75° (e.g., between about 1° and 5°, about 1° and 10°, about 1° and 15°, about 10° and 15°, about 15° and 25°, about 20° and 30°, about 25° and 35°, about 30° and 40°, about 35° and 45°, about 40° and 50°, about 45° and 55°, about 50° and 60°, about 55° and 65°, about 60° and 70°, or about 65° and)75°, e.g., between about 1° and 37.5°, about 10° and 40°, about 15° and 45°, about 22.5° and 67.5°, about 40° and 70°, about 25° and 75°, or about 35° and 75°, e.g., at least 5°, 10°, 15°, 20°, 25°, or 30°, e.g., about 22.5°, about 30°, about 45°, about 60°, or about 67.5°.

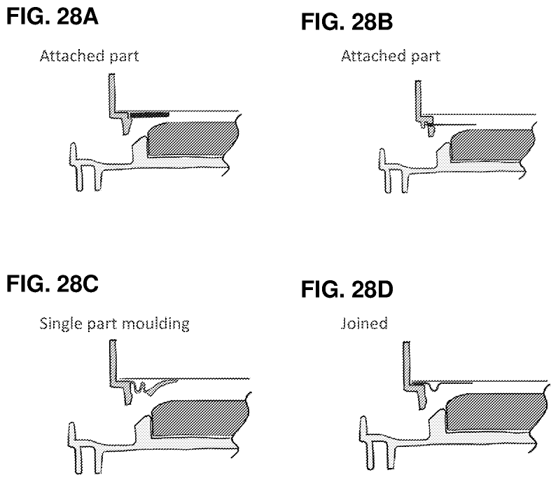

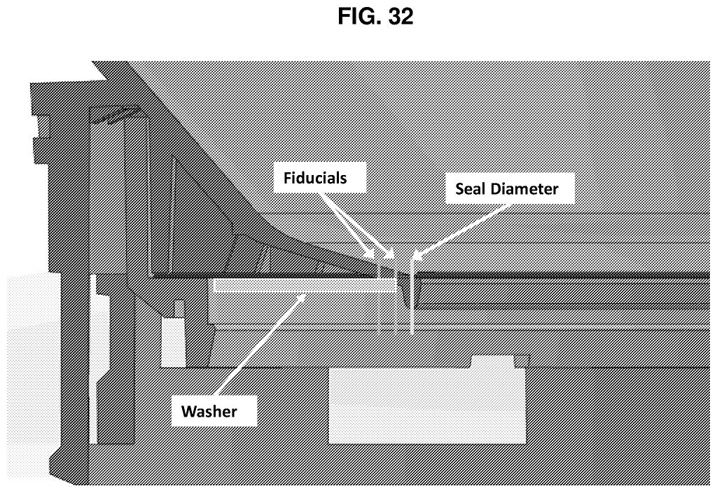

A filtration assembly may contain a washer disposed beneath the membrane, see, e.g., FIGS. 28A-28D and FIG. 32, e.g., around the inner edge of the membrane frame. A washer may be a single element, e.g., an annular ring or a partial ring (e.g., C-shaped) or a plurality of elements, e.g., arranged annularly as a discontinuous ring). A washer may be, e.g., plastic, e.g., Teflon, polypropylene, polyethylene, polyethylene terephthalate, polyester, polycarbonate, etc. A washer in the filtration assembly may be a part of, e.g., attached to (e.g., by mechanical engagement or sealing, e.g., by adhesive or thermal bonding), or formed with (e.g., molded, e.g., as a single part) the membrane frame. A washer may reduce bubble trapping under the membrane (e.g., when transferred to the cassette). A washer that is part of the membrane support may improve the flatness of the membrane, e.g., when stretched over the solid or semi-solid nutrient media. A washer of the invention may include fluorescent material to show through as a fiducial marking, e.g., via a hole in the membrane or membrane frame. A washer may be a thin film, e.g., less than about 0.2 mm thick, e.g., 0.1 to 0.2 mm, 0.5 to 0.15 mm, 0.01 to 0.05 mm, or about 0.05 mm, 0.07 mm, 0.1 mm, 0.11 mm, 0.12 mm, 0.13 mm, 0.14 mm, or about 0.15 mm thick. A washer may be a thicker, more rigid ring, e.g., greater than 0.2 mm thick, e.g., about 0.2 to 0.3 mm, 0.25 to 0.35 mm, 0.3 to 0.4 mm, 0.35 to 0.45 mm, 0.4 to 0.5 mm, 0.45 to 0.55 mm, 0.5 to 0.6 mm, 0.55 to 0.65 mm, 0.6 to 0.7 mm, 0.65 to 0.75 mm, 0. 7 to 0.8 mm, 0.75 to 0.85 mm, 0.8 to 0.9 mm, 0.85 to 0.95 mm, or 0.9 to 1 mm thick. A washer may be flat. A washer may be smooth. The washer may allow for a reduction in friction when the membrane is seated on a cassette base.

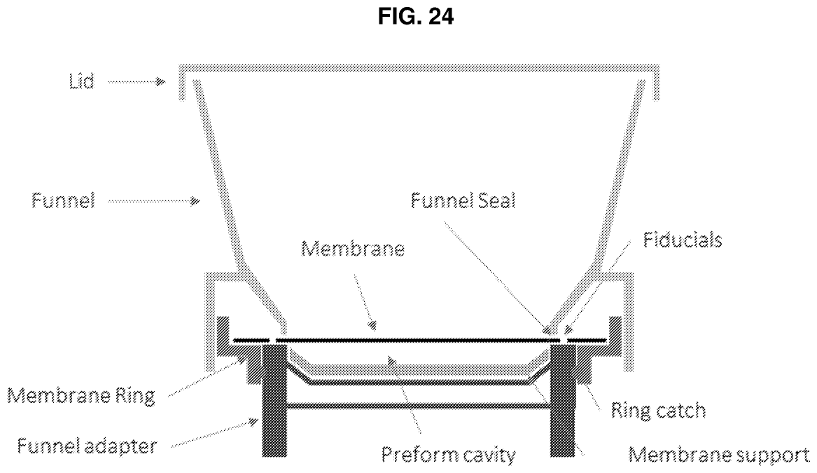

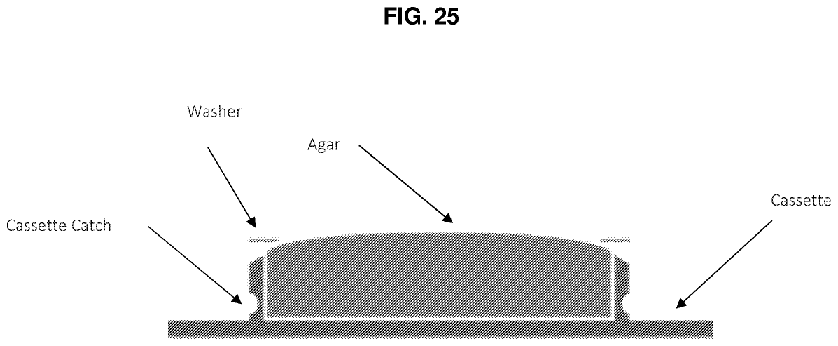

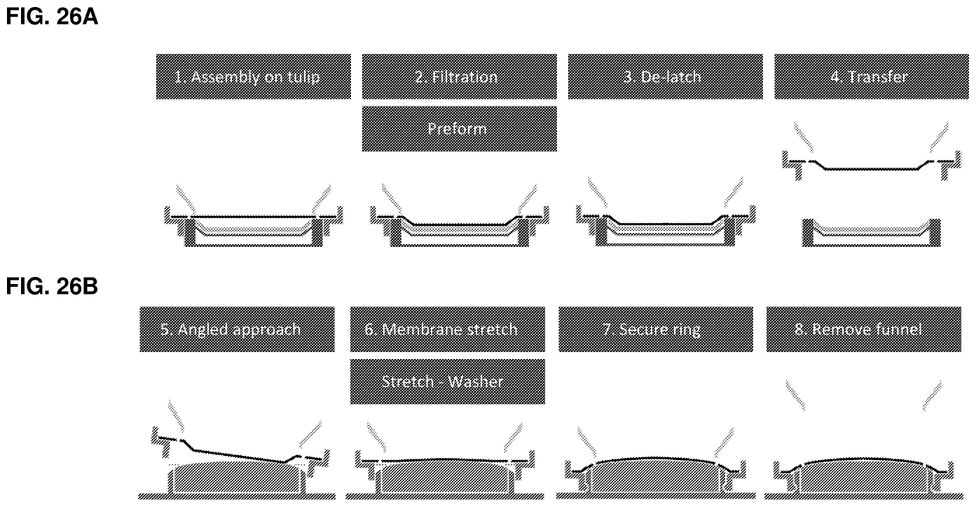

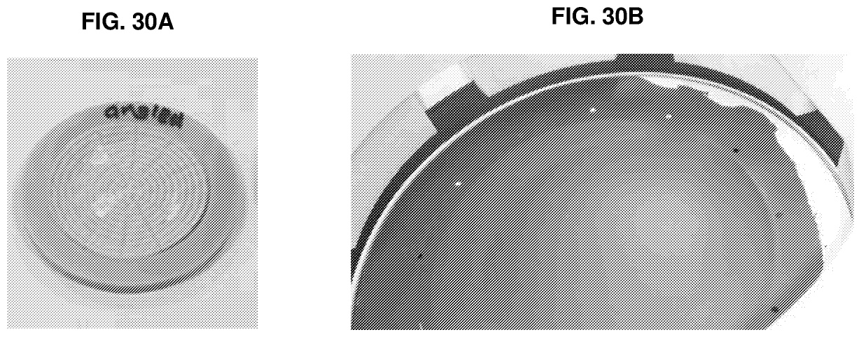

A filtration assembly of the invention may be configured to stretch the membrane such that it conforms to a flat surface (e.g., the surface of the membrane support), see, e.g., FIGS. 24 and 26A. A filtration assembly may include a cavity between the membrane support and the membrane, into which the membrane is drawn when filtration occurs. The membrane support may be shaped to create a cavity, e.g., having edges that taper upwards around a central flat disc area. An interior of the base may be similarly tapered to accommodate or create the cavity. A shim in the base may shape the cavity. The shape of the membrane after filtration may be determined in whole or in part by a shim (see, e.g., FIGS. 30A and 30B). The shim may be an addition to (e.g., resting on or attached to, e.g., by mechanical engagement or sealing, e.g., by adhesive or thermal bonding), or part of (e.g., molded with), the filtration base.

### Cassettes

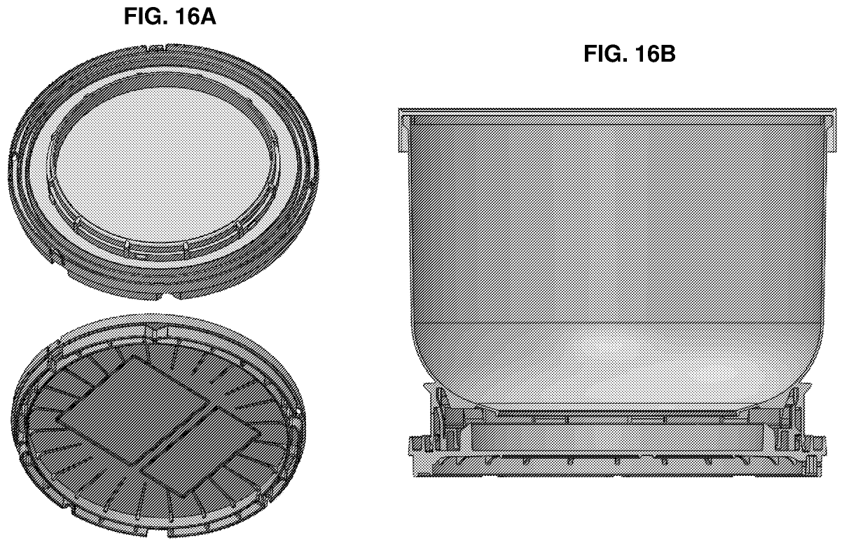

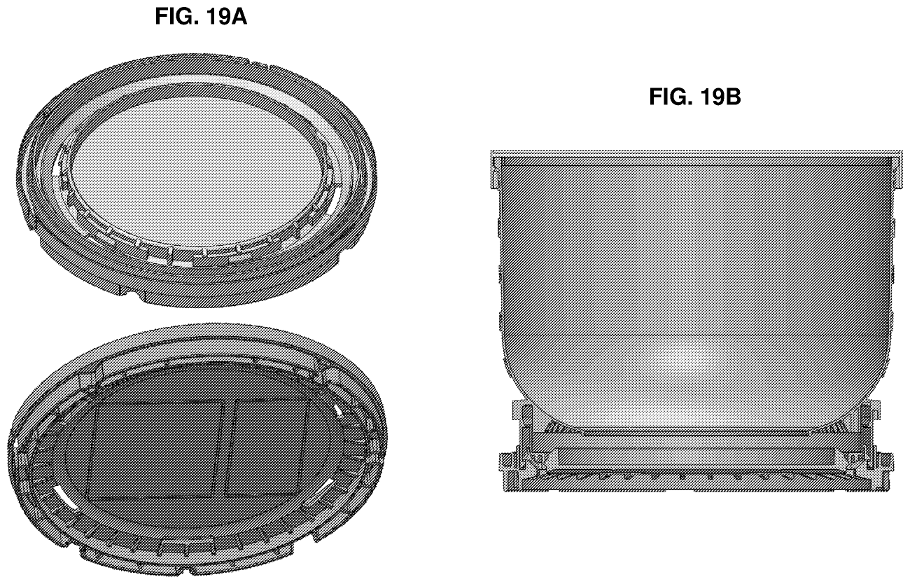

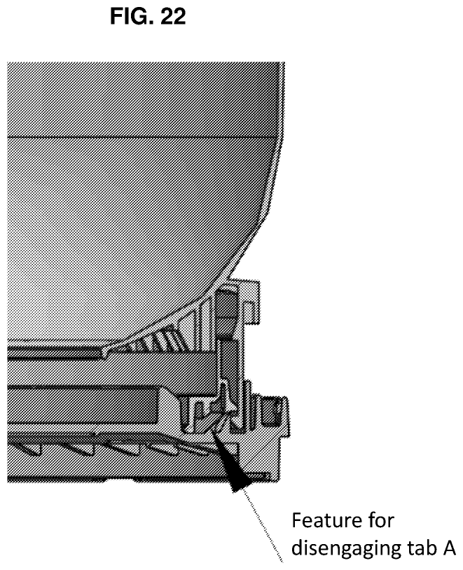

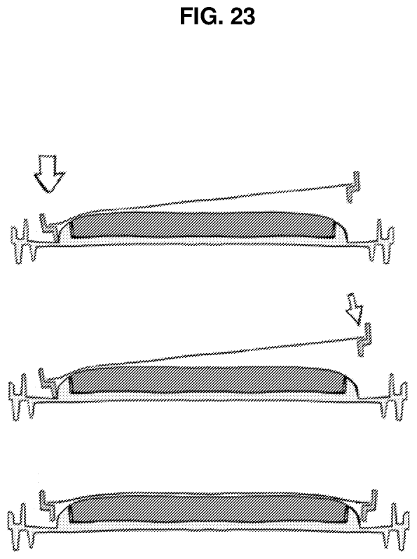

The invention provides cassettes for cell growth, e.g., on the membrane described herein. A cassette of the invention includes a cassette base (see, e.g., FIG. 3, FIG. 16A, or FIG. 19A), with a base layer that includes an outer wall, a first inner wall including a plurality (e.g., 2, 3, 4, 5, 6, 7, 8, 9, or 10 or more) of radially disposed gaps, and a second inner wall. The first inner wall is located between the second inner wall and the outer wall. The second inner wall defines a well for holding a solid or semi-solid nutrient media. The second inner wall also includes an outside ledge, which can allow the well to be over-filled with nutrient media. The solid or semi-solid media having a flat growth area that is higher than second inner wall ensures that the membrane is kept flat for imaging by pressing the membrane conformally against the media when the frame is attached to the base. The cassette base layer can also have one or more holes (e.g., 2, 3, 4, 5, 6, 7, 8, 9, or 10 or more) between the first inner wall and the outer wall (e.g., FIG. 3 or FIG. 19A). These holes can help prevent air bubbles being trapped under the membrane when it is attached to the cassette base. Air bubbles trapped between the membrane and media may not only prevent areas of the membrane from receiving nutrients but can also distort the membrane and confound imaging. The holes may also receive one or more tabs on the membrane frame as part of a locking mechanism (see, e.g., FIG. 19B) between the membrane frame and cassette base. The cassette base may include features (see, e.g., FIG. 21 and FIG. 22) which act to disengage interlocking features of the membrane frame and funnel when the funnel and membrane frame are pushed onto the cassette base. The cassette also includes a cassette lid releasably sealable to the base.

In some embodiments, the second inner wall can include a plurality of (e.g., 2, 3, 4, 5, 6, 7, 8, 9, or 10 or more) supports protruding radially towards the first inner wall. Such radial supports can help in holding an over filled media to the ledge and increase the diameter of the flat growth area. Other inner walls may also feature a plurality of supports that protrude radially towards the outer wall, e.g., to support the membrane frame.

The cassette may also have a third inner wall disposed between the first inner wall and the outer wall and may additionally have a fourth inner wall between the third inner wall and the outer wall (see, e.g., FIG. 3). Cassettes may include additional inner walls (e.g., 5, 6, 7, 9, or 10 or more). In some embodiments, one or more of the inner walls is configured to interact with a locking mechanism between the membrane frame and the funnel (e.g., a tab) in order to disengage the locking mechanism and release the funnel.

The solid or semi-solid nutrient medium may be any suitable medium. Examples include Sabouraud dextrose agar (SDA), R2A agar, tryptic soy agar (TSA) letheen, and plate count agar (PCA). Other media are known in the art. The flat growth area is typically at least 5 mm in diameter, e.g., 5 to 200 mm, e.g., 10 to 80 mm.

The cassette lid may be thermoformed, or injection molded. The cassette lid may preferably be optically transparent (e.g., to detect cells by light) and/or non-fluorescent (e.g., to allow for detection of cells by autofluorescence), for example, a cyclic olefin polymer lid. Other suitable transparent lid materials may include PET, polymethylmethacrylate, ethylene tetrafluoroethylene, polystyrene, etc. The cassette may include a cover lid to protect the cassette during shipping, e.g., a flexible polymer (e.g., rubber) lid.

The cassette base may preferably be made of a non-fluorescent material to prevent interference with imaging, e.g., black styrene-butadiene-copolymer.

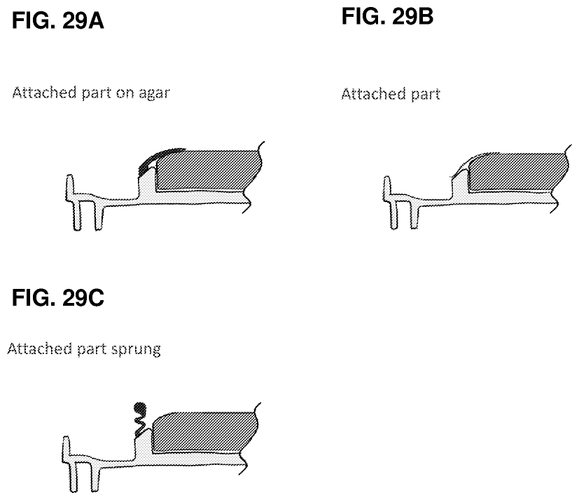

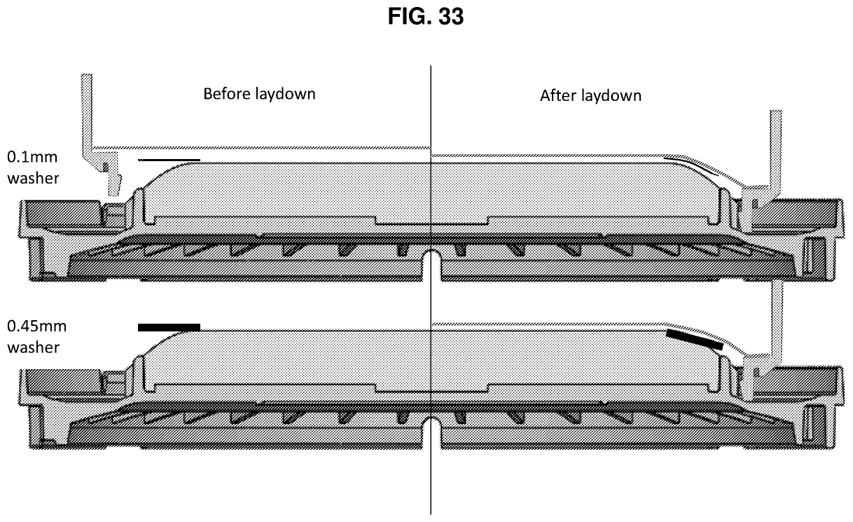

Cassettes may include a washer on top of or connected to the second inner wall, see, e.g., FIGS. 29A-29C and FIG. 33, e.g., disposed to be between the membrane and the second inner wall and/or the solid or semi-solid nutrient media. A washer in the cassette may be a single element, e.g., an annular ring or a partial ring (e.g., C-shaped) or a plurality of elements, e.g., arranged annularly as a discontinuous ring). A washer may be, e.g., plastic, e.g., Teflon, polypropylene, polyethylene, polyethylene terephthalate, polyester, polycarbonate, etc.). A washer in the cassette may be a part of, e.g., attached to (e.g., by mechanical engagement or sealing, e.g., by adhesive or thermal bonding), or formed with (e.g., molded, e.g., as a single part) the cassette base. A washer may reduce bubble trapping between the membrane and the solid or semi-solid nutrient media. A washer may improve the flatness of the membrane, e.g., when stretched over the solid or semi-solid nutrient media. A washer that is part of the cassette base may improve the flatness of the membrane, e.g., when stretched over the solid or semi-solid nutrient media. A washer of the invention may include fluorescent material to show through as a fiducial marking, e.g., via a hole in the membrane or membrane frame. A washer may be a thin film (see, e.g., 29B), e.g., less than about 0.2 mm thick, e.g., 0.1 to 0.2 mm, 0.5 to 0.15 mm, 0.01 to 0.05 mm, or about 0.05 mm, 0.07 mm, 0.1 mm, 0.11 mm, 0.12 mm, 0.13 mm, 0.14 mm, or about 0.15 mm thick. A washer may be a thicker (see, e.g., FIG. 29A), more rigid ring, e.g., greater than 0.2 mm thick, e.g., about 0.2 to 0.3 mm, 0.25 to 0.35 mm, 0.3 to 0.4 mm, 0.35 to 0.45 mm, 0.4 to 0.5 mm, 0.45 to 0.55 mm, 0.5 to 0.6 mm, 0.55 to 0.65 mm, 0.6 to 0.7 mm, 0.65 to 0.75 mm, 0. 7 to 0.8 mm, 0.75 to 0.85 mm, 0.8 to 0.9 mm, 0.85 to 0.95 mm, or 0.9 to 1 mm thick. A washer may be flat. A washer may be smooth. A washer may be a sprung, attached part (see, e.g., FIG. 29C), e.g., disposed to flex down toward the nutrient media under pressure of the pressed down membrane. When a washer is a thin film (e.g., about 0.1 mm thick), it may conform to the shape of the membrane and/or nutrient media. When thicker, e.g., about 0.45 mm thick, the washer may conform to the pressure of the washer and take on a Belleville washer shape, digging into the nutrient media. When the washer is thicker, e.g., about 0.8 mm, it may conform lesser to the membrane pressure and digs into the nutrient media. The washer may allow for a reduction in friction when the membrane is seated on a cassette base.

The cassette base may be configured to engage with the membrane frame such that the membrane frame and membrane are angled (e.g., between 1° and 75°) as the membrane makes contact with the solid or semi-solid nutrient media. For example, the cassette base may include angled features such as an angled slot, tab, catch, cam, latch, or hook that engages the membrane frame at an angle relative to the surface of the medium.

The cassette base may be configured (e.g., by having positioning features) so that the membrane frame may be initially lowered onto the base at an angle between 1° and 75° (e.g., between about 1° and 5°, about 1° and 10°, about 1° and 15°, about 10° and 15°, about 15° and 25°, about 20° and 30°, about 25° and 35°, about 30° and 40°, about 35° and 45°, about 40° and 50°, about 45° and 55°, about 50° and 60°, about 55° and 65°, about 60° and 70°, or about 65° and)75°, e.g., between about 1° and 37.5°, about 10° and 40°, about 15° and 45°, about 22.5° and 67.5°, about 40° and 70°, about 25° and 75°, or about 35° and 75°, e.g., at least 5°, 10°, 15°, 20°, 25°, or 30°, e.g., about 22.5°, about 30°, about 45°, about 60°, or about 67.5°.

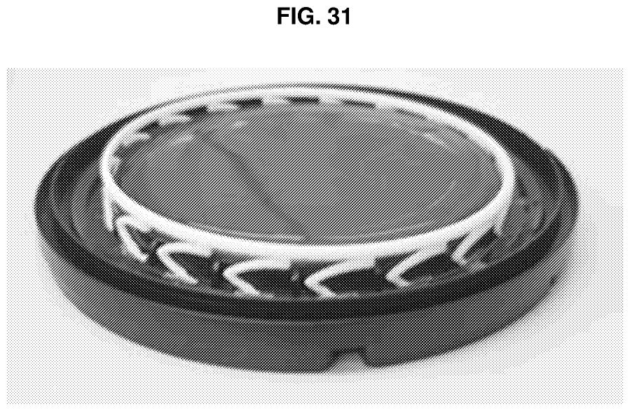

In some embodiments, the cassette base may include a ring with springs of different heights, see, e.g., FIG. 31, e.g., disposed such that one side of the ring contacts the membrane or membrane frame before its antipode, thereby causing the opposite side of the membrane to contact the nutrient media first.

The cassette base, together with the membrane frame and solid or semi-solid media may act to stretch the membrane when the membrane frame is attached to the cassette base. A washer of the invention may also help in stretching the membrane.

### Systems and Kits

The invention provides systems for filtering and culturing cells, e.g., for microbiological enumeration. Systems of the invention include a filtration assembly and a cassette of the invention. In systems of the invention, the membrane frame of the filtration assembly is configured to detach from the base of the filtration assembly, attach to the cassette base, and to detach from the funnel once attached to the cassette base. By keeping the membrane frame attached to the funnel until it is securely attached to the cassettes base, the membrane is protected from contamination and damage during the transfer process.

Systems of the invention require the membrane frame to be attachable to the cassette base in a way that creates a stronger attachment than that of the membrane frame to the funnel. To achieve this, the membrane frame may include a protruding ring, and the cassette base may include tabs than interlock with the protruding ring to attach the membrane frame to the cassette base. Alternatively, the features may be reversed, and the cassette may feature a protruding ring, e.g., from one of the inner walls, and the membrane ring may include tabs. Alternatively or in addition, the cassette may include features (e.g., an inner ring, as shown in FIG. 22), which press against tabs on the membrane frame or funnel (see, e.g., FIG. 21) and disengages the interlocking features.

In some embodiments, attaching the membrane frame to the cassette base places the membrane in conformal contact with the growth area, i.e., keeps the membrane and media pressed together. Maintaining the flatness of the membrane growth area, e.g., by pressing against the media, is advantageous during imaging.

Kits may include one or more of the devices described herein. For example, when the funnel and/or cassette do not include a lid, a kit of the invention can include one or more separate lids for covering the funnel and or cassette. Lids may be included in a kit with the filtration assembly and cassette or packaged separately. Kits may include both a filtration assembly and a cassette, or multiple filtration assemblies, or multiple cassettes, so that, for example, a microorganism-agnostic filtration assembly may be paired with a microorganism specific cassette by the user. Alternatively, the filtration assembly may be microorganism specific. A kit may include a flexible polymer cover to protect the cassette base during shipping and a separate lid that is transparent and/or non-fluorescent for incubation and imaging. Kits may also include a washer as described herein as a separate component.

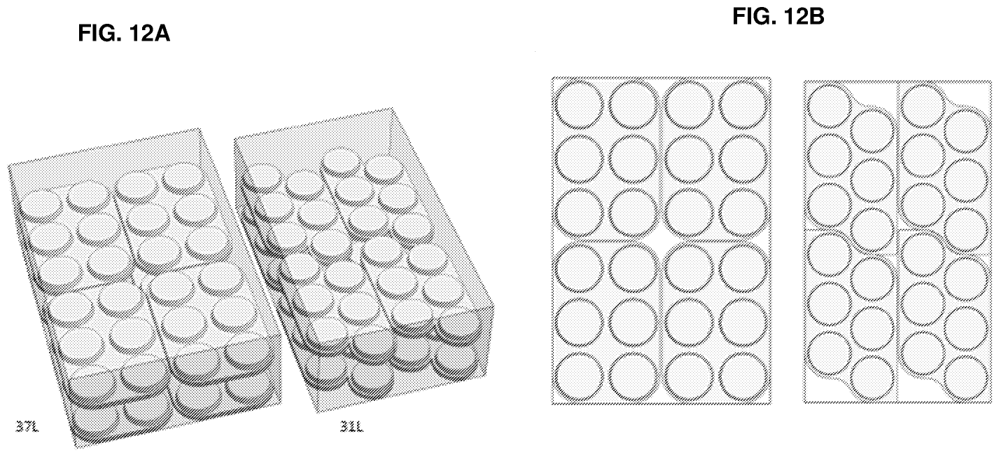

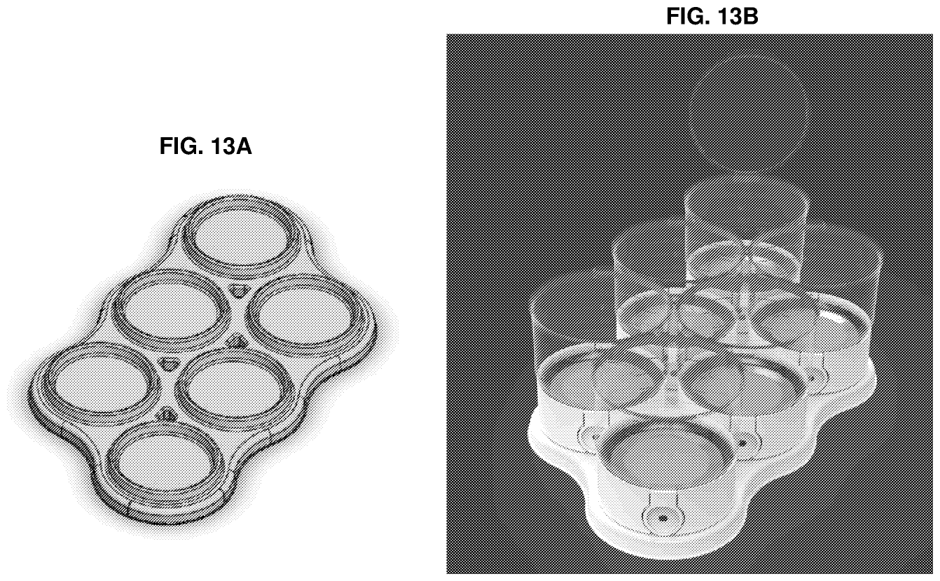

Systems or kits of the invention may also include a tray for holding multiple filtration assemblies (e.g., 2, 3, 4, 5, 6, 7, 8, 9, or 10 or more). In some embodiments, the system also includes a base disposed to hold six filtration assemblies as shown, for example, in FIG. 13A and FIG. 13. A tray may be stackable with and without the filtration assemblies. A tray may hold the filtration assemblies in a staggered layout (see, e.g., FIG. 13A, and FIG. 13B) or a rectangular layout (e.g., FIG. 12B). A staggered arrangement can allow stacked trays of filtration assemblies to take up less volume, e.g., during shipping, than a rectangular arrangement (see, e.g., FIG. 12A). Trays may be thermoformed, or injection molded.

The filtration assemblies and cassettes of the invention may be combined with various external components, e.g., vacuum pumps, aspirators, liquid handling robots, robotic arms, light sources (e.g., lasers), detectors, heaters (e.g., for incubation), coolers (e.g., for storage), reagents (e.g., for cell staining) etc. as parts of kits and systems.

### Methods

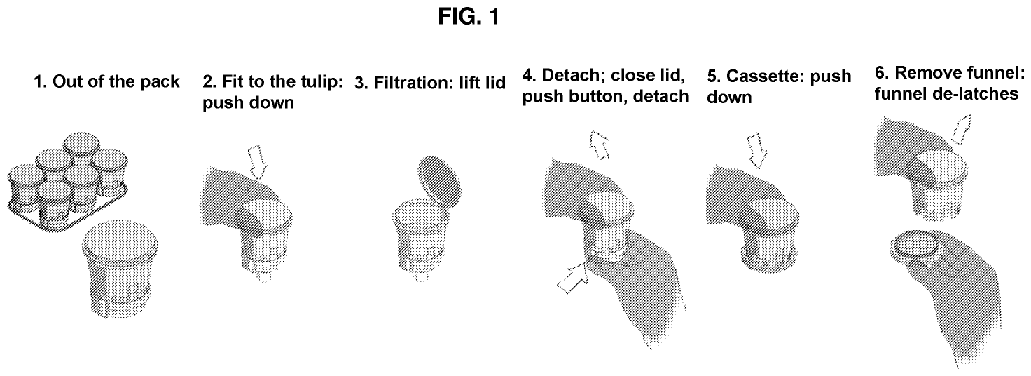

The invention provides methods for determining the presence of microorganisms (e.g., bacteria or yeast) in a sample, e.g., a water sample. An exemplary method includes first attaching the filtration assembly to a source of vacuum, e.g., by connecting the base to a tulip valve connected to a vacuum pump or aspirator. The method then involves flowing a sample fluid through the filtration assembly, such that any cells in the fluid are retained on the membrane. The funnel attached to the membrane frame is then detached from the base (e.g., by pressing a button that releases a latch), before attaching the membrane frame to a cassette base of the invention (e.g., as shown in FIG. 16B or FIG. 19B). The funnel is then detached from the membrane frame and the cassette incubated to allow any retained cells to multiply. The method advantageously allows transfer of the membrane without handling and risking damage or contamination. The method is particularly advantageous for incorporation into an automated testing system. An example of a method of the invention is shown in FIG. 1.

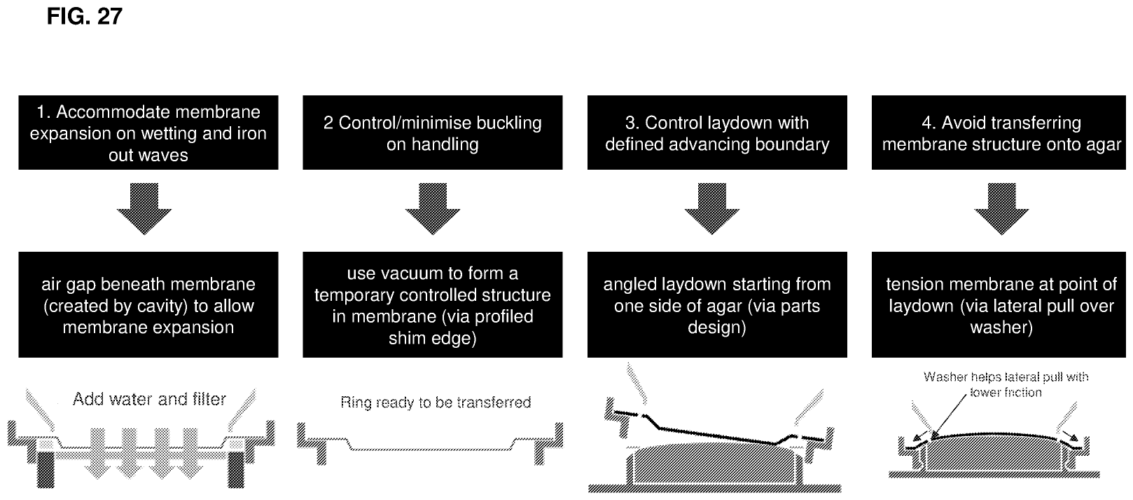

Methods of the invention may include using pressure, e.g., from vacuum, to cause the membrane to take on the shape of a cavity between the membrane and membrane support (see, e.g., FIGS. 24, 26A, and 27). The membrane may maintain this shape after detaching the membrane frame from the base and until the membrane frame is attached to the cassette base (see, e.g., FIG. 27). The shaped membrane may interact with the cassette (e.g., the solid or semi-solid media) to prevent bubble trapping during attachment of the membrane from to the cassette base (see, e.g., FIG. 27).

In methods of the invention, the membrane may make a first contact with the flat growth area at a first point on its circumference and a final contact at a second point on the circumference that is antipodal to the first point (see, e.g., FIG. 26B). The propagation of contact between the first contact point and final point at a continuously diminishing angle of contact can act to minimize or eliminate, e.g., bubble trapping and wrinkling. The membrane frame may be initially lowered onto the base at an angle between 1° and 75° relative to the flat growth area (e.g., between about 1° and 5°, about 1° and 10°, about 1° and 15°, about 10° and 15°, about 15° and 25°, about 20° and 30°, about 25° and 35°, about 30° and 40°, about 35° and 45°, about 40° and 50°, about 45° and 55°, about 50° and 60°, about 55° and 65°, about 60° and 70°, or about 65° and)75°, e.g., between about 1° and 37.5°, about 10° and 40°, about 15° and 45°, about 22.5° and 67.5°, about 40° and 70°, about 25° and 75°, or about 35° and 75°, e.g., at least 5°, 10°, 15°, 20°, 25°, or 30°, e.g., about 22.5°, about 30°, about 45°, about 60°, or about 67.5°. Asymmetrically disposed locking features (e.g., disposed at different heights, or with different resistances) may be used to direct the angle of approach of the membrane frame (and membrane) during attachment of the membrane frame to the cassette base. The cassette and/or membrane frame may include a feature (e.g., an angled slot, tab, catch, cam, latch, hook, etc., or an attached or inserted asymmetrically sprung ring) that position the membrane to contact the flat growth area such that contact propagates from a first point to a final, antipodal point (e.g., at an initial angle between about 1° and 75°, e.g., with a diminishing angle) when the membrane frame is lowered onto the cassette base. A sprung ring may be a ring supported by an annularly arranged plurality of springs, where a highest spring of the plurality of springs is antipodal to a lowest spring of the plurality and springs of the plurality disposed therebetween are tapered in height.

The method may further include imaging the membrane to detect any colonies formed from the retained cells. For example, by detecting the autofluorescence of certain microorganisms, including small colonies of only a few hundred cells. Imaging may also involve monitoring the growth of colonies by taking a time series of images, for which embodiments of the invention featuring fiducial markings are particularly appropriate. The cassettes may be advantageously imaged in the Growth Direct® automated microbial detection system (Rapid Micro Biosystems).

Other embodiments are in the claims.

1.-45. (canceled)

46. A filtration assembly, comprising:
a) a funnel;
b) a membrane frame comprising a porous membrane;
c) a base comprising a membrane support and an outlet;
wherein the membrane frame is releasably attached to the funnel and the base is releasably attached to the funnel; wherein, during filtration, liquid flows from the funnel through the membrane and membrane support to the outlet; and wherein the membrane support keeps the membrane flat during filtration.

47. The filtration assembly of claim 46, wherein the funnel is attached to the base by a locking mechanism that releases when a portion of an exterior wall of the base is pressed.

48. The filtration assembly of claim 46, wherein the porous membrane is ultrasonically bonded or heat staked to the membrane frame.

49. The filtration assembly of claim 46, wherein the membrane frame is releasably attached to the funnel by an interlocking arrangement of a raised lip on the funnel and a recess in the membrane frame.

50. The filtration assembly of claim 46, wherein the membrane frame comprises a protruding ring that interlocks with tabs on a cassette base.

51. The filtration assembly of claim 46, further comprising a funnel lid that covers an opening to the funnel.

52. The filtration assembly of claim 46, wherein the membrane support is attached to the filter base.

53. The filtration assembly of claim 46, wherein the filter base comprises a plurality of supports configured to promote fluid flow to the outlet or to support the membrane support.

54. The filtration assembly of claim 46, wherein the filtration assembly further comprises a washer.

55. The filtration assembly of claim 54, wherein:
a) the washer is attached to the membrane support or is a thin film; or
b) the washer and membrane support are a single molded part; or
c) the washer and membrane support are a single molded part.

56. The filtration assembly of claim 54, wherein the washer comprises a spring.

57. The filtration assembly of claim 46, further comprising a cavity or a shim between the membrane and membrane support.

58. The filtration assembly of claim 57, wherein the membrane support or the base are shaped to create the cavity.

59. A system for filtering and culturing cells, comprising:
the filtration assembly of claim 1; and
a cassette, comprising:
a) a cassette base, comprising:
i. a base layer comprising an outer wall, a first inner wall comprising a plurality of radially disposed gaps, and a second inner wall comprising an outside ledge and defining a well, wherein the first inner wall is disposed between the outer wall and the second inner wall;
ii. solid or semi-solid nutrient media disposed in the well and having a flat growth area, wherein the growth area is higher than the second inner wall; and
b) a cassette lid releasably sealable to the base;
wherein the membrane frame is configured to detach from the filter base with the funnel, attach to the cassette base, and to detach from the funnel once attached to the cassette base.

60. The system of claim 59, wherein attaching the membrane frame to the cassette base places the membrane in conformal contact with the flat growth area.

61. A method for determining the presence of microorganisms, comprising:
a) attaching the filtration assembly of any one of claim 1 to a source of vacuum;
b) flowing a sample fluid through the filtration assembly, wherein any cells in the fluid are retained on the membrane;
c) detaching the funnel attached to the membrane frame from the base;
d) attaching the membrane frame to a cassette base;
e) detaching the funnel from the membrane frame; and
f) incubating the cassette.

62. The method of claim 61, further comprising imaging the membrane to detect any colonies formed from the retained cells.

63. The method of claim 61, wherein the filtration assembly comprises a cavity and step (a) comprises applying pressure to cause the membrane to conform to a shape of the cavity.

64. The method of claim 61, wherein during step (d) the membrane frame is initially lowered onto the base at an angle between 1° and 75° relative to the flat growth area.

65. The method of claim 61, wherein the cassette comprises a feature that engages the membrane frame to position the membrane to contact the flat growth area at an angle between 1° and 75° relative to the flat growth area as the membrane frame is lowered onto the cassette base.
